(19) United States
(12) Patent Application Publication
(10) Pub. No.: US 2006/0241782 A1
(43) Pub. Date: Oct. 26, 2006

(54) Vertical shock absorbing prosthetic foot

(76) Inventor: Michael J. Curtis, Green Bay, WI (US)

Correspondence Address: NICHOLAS A. BRANNEN, 104 SOUTH MAIN STREET, SUITE 300, FOND DU LAC, WI, 54935, US

(21) Appl. No.: 11/111,443

(22) Filed: Apr. 21, 2005

(51) Int. Cl. A61F 2/66; A61F 2/62

(52) U.S. Cl. 623/55; 623/38

(57) ABSTRACT

The present invention relates to a vertical shock absorbing prosthetic foot that has a forefoot having a toe spring and a toe plate and a heel having a heel spring and a heel strike. The heel spring and toe spring comprise a foot spring. The toe spring can be forward opening and the heel spring can be rearward opening. The toe spring can be located rearward of the heel spring. The foot spring can be generally loop shaped. A connector is used to connect the foot to a residual limb or other prosthetic components. The connector can have a top piece and a bottom piece, and the connector has a shape that is complementary to the foot spring. The connector can be adjustably connected to the foot spring by rotating the connector around the foot spring to adjust the location of the heel strike.

### BACKGROUND OF THE INVENTION

1. Field of the Invention

The present invention relates to a prosthetic foot, and more particularly to a vertical shock absorbing prosthetic foot comprising a foot spring for absorbing energy, that is flexible about many axis, that is efficient at storing and returning energy during use and that is angularly adjustably connectable to a residual limb.

2. Description of the Related Art

People frequently are in need of prosthetic limbs as a result of accidents, disease or birth defects. The need for quality prosthetic feet is high. There have been many attempts to make suitable prosthetic feet, each desiring to reach goals of safety, functionality and comfort, among other qualities. The existing prosthetic feet have achieved varying levels of success at attaining each of these stated goals. Further, many strategies have been utilized in designing prosthetic feet. Some designs are relatively noncomplicated, and seek to use a simple design. Other designs are complex, and seek to simulate or copy the structure of the human foot.

One prosthetic foot in particular is made by Freedom Innovations and sold under the name Renegade LP. This foot has an uninterrupted component spanning from the ankle to the toe. A second component extends from the heel to the front of the foot. While this foot may work well for its intended purpose, it is not without some limitations. For example, there is not a heel spring incorporated into the design. Another limitation of this foot is that there is no way to adjust the location of the heel with respect to the residual limb.

Another existing prosthetic foot is sold by Ossur under the name Modular III. This foot has one uninterrupted component forming the ankle and spanning to the toe area. A heel component depends rearward from the major component to the heel. This heel component connects to the bottom surface of the major component. A primary advantage of this foot resides in its simplicity. However, there are drawbacks also associated therewith. One limitation is the lack of adjustability of the location of the heel with respect to the residual limb. A further limitation of this foot is the lack of a heel spring.

A third prosthetic foot is an assembly that is shown in U.S. Pat. No. 6,129,766 to Johnson et al. This patent discloses an ankle member, a heel member pivotally connected to the ankle member, and an elongate metatarsal-toe member having a rear portion underlying a forward portion of the heel member and projecting forwardly from the heel member. This foot has compressible elements incorporated between the pivotally connected members. This foot, being an assembly, is relatively complicated when compared to the previously described prosthetic feet. A further limitation of this foot is that there may be inherent noise problems incorporated into the design of the compressible elements. This is evidenced by the presence of, and the need for, noise abatement features being described in the disclosure.

A fourth prosthetic foot is sold by Otto Bock under the name LuXon Max. This foot shows a relatively flat and uninterrupted component spanning from the ankle location forward to a location above and rearward of the toe location. Distinct heel and toe components are connected to the bottom of the flat component. One limitation of this foot is that there is no way to adjust the location of the heel with respect to the residual limb. Another limitation of this foot is that there is no heel spring incorporated into the design.

Yet another prosthetic foot is shown in U.S. Pat. No. 6,602,295 to Doddroe et al. The foot shown in this patent has a foot plate, which is an uninterrupted plate spanning from heal to toe. A toe spring and a heel spring are provided. The toe spring is forward of the heel spring. The toe spring and heel spring are independently connected to a top plate. Further, the heel spring is rotatably connectable to the top plate and the foot plate.

Another prosthetic foot is shown in U.S. Pat. No. 6,241,776 to Christensen. The foot shown in this patent has a forefoot reinforcement member extending from an attachment section, through a curvilinear spring and arch section, to a toe end. A heel member extends from the arch section to the heel end. The strength and energy return in this foot is due to the flexing of the members. A limitation of this foot is that it is lacking is adjustability. A further limitation is that it does not have a heel spring.

Another prosthetic foot is shown in U.S. Pat. No. 5,037,444 to Phillips. That patent shows a foot with a forefoot portion and a demountably connected heel portion. At toe-off, the energy return is created from energy stored during the flexing of the forefoot member. One limitation is that the foot of this invention does not have a heel spring. A further limitation is the lack of adjustability of the location of the heel.

Thus there exists a need for a prosthetic foot that solves these and other problems.

### SUMMARY OF THE INVENTION

The present invention relates to a vertical shock absorbing prosthetic foot that has a forefoot having a toe spring and a toe plate and a heel having a heel spring and a heel strike. Advantageously, the heel spring and toe spring comprise a foot spring. These components can be made of carbon epoxy composite material and can be integral, or can be assembled from multiple components. A connector is used to connect the foot to a residual limb or other prosthetic components. The connector can have a top piece and a bottom piece.

According to one aspect of the present invention, the toe spring can be a forward opening, or forward facing, toe spring, and the heel spring can be a rearward opening heel spring. The toe spring can have a rear that is rearward of the front of the heel spring. The foot spring, being comprised of the toe spring and heel spring, can be generally loop shaped. The connector has a shape that is complementary to the foot spring.

According to another aspect of the present invention, the connector can be adjustably connected to the foot spring by rotating the connector around the foot spring. This results in the practitioner being able to adjustably select the location of the heel with respect to the residual limb.

One advantage of the prosthetic foot of the present invention is that the connector is adjustably connectable to the foot, allowing the practitioner to adjust the location of the heel with respect to the connector. The location of the heel with respect to the connector contributes to overall length of the prosthetic limb, and can affect gate. Further, varying the location of the heel will affect the amount of flexing and deflection of the heel strike and compression of the heel spring at heel-strike. This adjustability results in the foot being more properly positioned given the needs and uses of a particular person.

Another advantage of the prosthetic foot of the present invention is that the foot spring is comprised of the toe spring and the heel spring. In this regard, the toe spring and heel spring are coacting springs that are not entirely independent of each other. Further, the toe spring, or at least a portion thereof, is located rearward of at least a portion of the heel spring. This advantageously allows for increased space for spring compression of the toe spring and heel spring in the limited space of the prosthetic foot.

Related, a further advantage of the prosthetic foot of the present invention is that the present invention provides support to the person at mid-stance. This is accomplished by flexing of the heel strike and toe plate, and of compression of the heel spring and toe spring. This flexing and compression relieve problems that may normally be associated with flat foot. Further, the energy in the heel strike and heel spring is useful in propelling the foot towards toe-off, and, alternatively, the energy in the toe plate and toe spring can be useful in assisting the person onto the heel strike if the person chooses to rock backwards.

Also related, a further advantage yet of the prosthetic foot of the present invention is that the present invention is free of sharp angles. Sharp angles in a prosthetic foot can lead to stress concentrations. Avoiding stress concentrations decreases the likelihood of failure of the foot.

A still further advantage yet of the present invention is that it is customizable to suit the specific needs of a given person. This is initially accomplished by selecting an appropriate blank foot from a given number of sizes of blank feet. The prosthetic foot can then be reduced in size from the initial size to a desired size.

A still further advantage yet of the prosthetic foot of the present invention is that it incorporates a split toe design. The toes can deflect and flex independent of each other, such as when an object is under only one of the two toes. This is advantageous for stability on terrain that is not completely flat. The split toe design is also advantageous during inversion and eversion to maximize the amount to forefoot that is in contact with the ground.

A still further advantage yet of the prosthetic foot of the present invention is that the foot can be tapered. The tapered design is advantageous in as much as it allows for the pre-selection of the flexural characteristics of the prosthetic foot.

A still further advantage yet of the prosthetic foot of the present invention is that there are no moving or rotatably connected parts that may fail over time. Further, none of the parts of the present invention are in a rubbing engagement. The present invention is therefore free from undesired noises and is free from the need of noise abatement features.

Other advantages, benefits, and features of the present invention will become apparent to those skilled in the art upon reading the detailed description of the invention and studying the drawings.

### BRIEF DESCRIPTION OF THE DRAWINGS

FIG. 1 is a side view of a preferred embodiment of the prosthetic foot of the present invention showing a connector connected to the foot.

FIG. 2 is a partial side view of a preferred embodiment of the prosthetic foot of the present invention showing a connector connected to the foot.

FIG. 3 is an exploded side view of a preferred embodiment of the prosthetic foot of the present invention showing a connector connected to the foot.

FIG. 4 is a side view of a preferred embodiment of the prosthetic foot of the present invention without a connector connected to the foot.

FIG. 5 is a top view of the prosthetic foot shown in FIG. 4

FIG. 6 is a rear view of the prosthetic foot shown in FIG. 4

FIG. 7 is a front view of the prosthetic foot shown in FIG. 4

FIG. 8 is an exploded side view of a preferred connector.

FIG. 9 is a top view of the connector shown in FIG. 8.

FIG. 10 is a top view of an alternative preferred connector.

FIG. 11 is a top view of an alternative preferred connector.

FIG. 12 is a side view of a preferred embodiment of the present invention showing the connector in a forward position on the foot.

FIG. 13 is a side view of a preferred embodiment of the present invention showing the connector in a rearward position on the foot.

FIG. 14 is a side view of the operation of a preferred embodiment of the prosthetic foot of the present invention at heel-strike.

FIG. 15 is a side view of the operation of a preferred embodiment of the prosthetic foot of the present invention at mid-stance.

FIG. 16 is a side view of the operation of a preferred embodiment of the prosthetic foot of the present invention at toe-off.

FIG. 17 is a side view of a preferred embodiment of the prosthetic foot of the present invention showing an object under a toe.

FIG. 18 is a cross-sectional side view of a preferred embodiment of the prosthetic foot of the present invention in an intended environment.

### DETAILED DESCRIPTION OF THE PREFERRED EMBODIMENTS

While the invention will be described in connection with several preferred embodiments, it will be understood that it is not intended to limit the invention to those embodiments. On the contrary, it is intended to cover all alternatives, modifications and equivalents as may be included within the spirit and scope of the invention as defined by the appended claims.

The prosthetic foot 10 of the present invention is preferably made from a carbon epoxy composite material. It will be understood that the present invention is not limited to being constructed of carbon epoxy composite material, and that other resilient materials can be used without departing from the broad aspects of the present invention.

The foot 10 is preferably made in blanks that can be reduced in length, or shortened, to meet the requirements of a particular person. Several size blanks can be made so that practitioners can select an appropriate blank for a starting point. Also, the blanks can be made to several heights or have features with varying sizes depending on the intended applications.

The prosthetic foot 10 of the present invention can be made to any desired thickness. Given that the thickness of the material is related to the flexural strength and amount of deflection of any given component of the foot, it is understood that blanks can be constructed having various thicknesses, and that each component within any given foot may be made with a different thickness. Further, it is understood that the thickness of material within any given part of the foot 10 can be tapered to achieve a desired flexural characteristic.

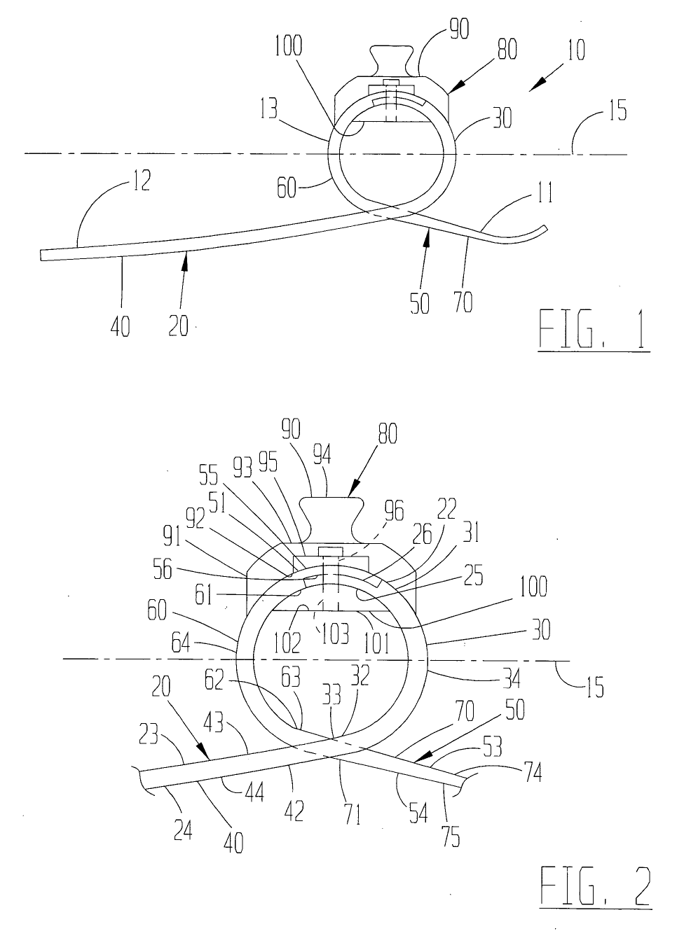

Calling attention now to FIGS. 1 and 18, it is shown that the prosthetic foot 10 of the present invention is intended for use with a connector 80 in order to be connected to another prosthetic component (not shown). The prosthetic foot 10 can be inserted into a shell 7 or cosmetic cover that resembles natural foot. The shell 7 can be used for direct contact with the ground 5 or for insertion into a shoe (not shown). It will be understood for the sake of clarity, the prosthetic foot 10 of the present invention is hereafter described in some circumstances as being in direct relation to the ground 5 or a surface without showing the shell 7.

A foot 10 is provided that is generally comprised of a rear portion 11 and a front portion 12. A foot spring 13 is provided between the front and rear of the foot 10. In the preferred embodiment, the foot spring 13 generally has a loop or circular shape. However, it will be understood that the foot spring 13 could have other shapes without departing from the broad aspects of the present invention. Further, the foot spring 13 preferably has a radius of approximately one inch. However, the foot spring 13 could have a larger or smaller radius without departing from the broad aspects of the present invention. The prosthetic foot 10 of the present invention has a longitudinal axis 15 spanning the length of the foot. The longitudinal axis 15 is generally parallel with the ground 5 when the foot rests on the ground during zero load conditions. When viewed from above, the prosthetic foot has a right side 16 and a left side 17.

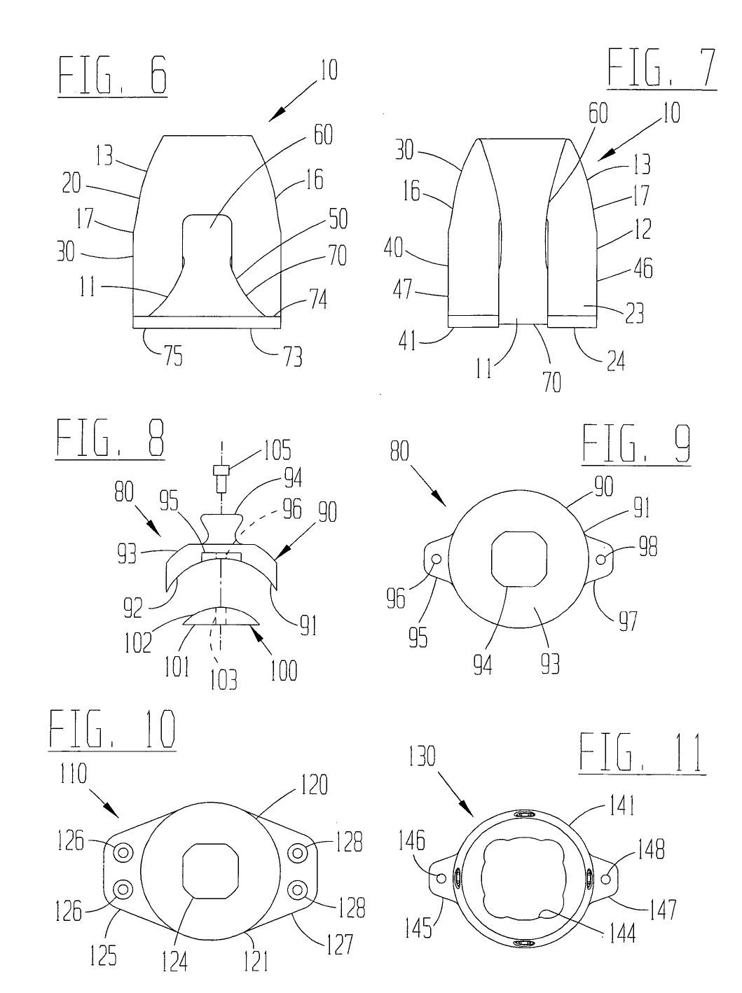

Referring now to FIGS. 4-7, several parts of a preferred embodiment of the prosthetic foot 10 of the present invention are more clearly shown. One part of the prosthetic foot 10 is the forefoot 20. The forefoot 20 has a first end 21 and a second end 22. The forefoot also has a first surface 23 and a second surface 24. The first surface 23 is on the bottom of the foot 10 at the first end 21, and is on the top at the second end 22. The second surface 24 is the opposite surface from the first surface 23 throughout the length of the forefoot 20. A plate 25 is at the second end 22 of the forefoot. The plate 25 has a mating surface 26. The plate 25 is preferably about a half as thick as the thickness of the forefoot 20 at the second end 22 of the forefoot.

A toe spring 30 is at the second end 22 of the forefoot 20. The toe spring 30 has a top 31, a bottom 32, a front 33 and a rear 34. The toe spring 30 generally has an undeflected shape of a semicircle. Toe spring 30 is a forward opening toe spring, or forward facing toe spring. The toe spring 30 could have a different shape without departing from the broad aspects of the present invention. During a downward load condition, the toe spring compresses and deforms from its nondeflected semicircular shape in response to the applied force. Conversely, during an upward load condition, the toe spring 30 expands and deforms in response to the applied force. The toe spring 30 can twist during inversion and eversion.

A toe plate 40 is at the first end 21 of the forefoot. The toe plate 40 has a front end 41 and a rear end 42. The front end 41 of the toe plate is the foremost portion of the prosthetic foot 10. The toe plate 40 has a top surface 43 and a bottom surface 44. The top surface 43 of the toe plate 40 coincides with the first surface 23 of the forefoot 20, and the bottom surface 44 of the toe plate coincides with the second surface 24 of the forefoot. The rear end 42 is preferably integral with the bottom front end of the toe spring 30. The body of the toe plate 40 flexibly depends forward and downwardly from the toe spring 30. The toe plate slightly curves upwardly along its length. During a downward load condition, the toe plate 40 flexes and deflects upwards in response to the applied force.

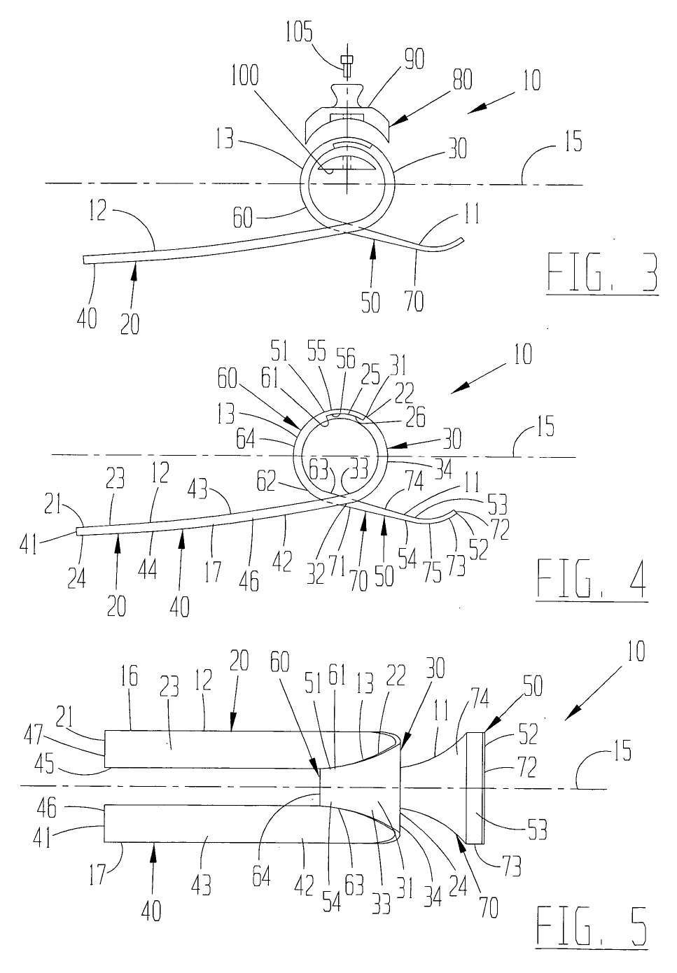

Looking now at FIG. 5 in particular and also FIG. 17, the toe plate 40 preferably has two toes 46 and 47, and are preferably separated by a slit 45 in the toe plate 40 having a selected width. Toe 46 is preferably on the left side 17 of the foot 10, and toe 47 is preferably on the right side 16 of the foot. The toes are shown to be approximately equal in width, and equally spaced from the longitudinal axis 15. However, the widths of the toes or their respective orientations could vary without departing from the broad aspects of the present invention. Toes 46 and 47 can flexibly deflect different amounts, or have independent deflection characteristics, depending on what terrain the person encounters. This is illustrated in FIG. 17, wherein an object is located under toe 47. Toe 47 correspondingly is upwardly flexibly deflected. Meanwhile, toe 46 is not flexibly deflected and contacts the ground 5. Both toes 46 and 47 will, however, deflect similarly when the load is not offset (such as during inversion or eversion) and when the person is on flat ground. During inversion and eversion condition (not shown), one side of each of the two toes 46 and 47 can remain in contact with the ground, while the opposite side or each respective toe is lifted off the ground.

Returning now to FIGS. 4-7, several preferred aspects of a heel 50 are illustrated. The heel 50 has a first end 51 and a second end 52. The heel 50 also has a first surface 53 and a second surface 54. The first surface 53 is on the bottom of the foot 10 at the second end 52, and is on the top at the first end 51. The second surface 54 is the opposite surface from the first surface 53 throughout the length of the heel 50. A plate 55 is at the first end 51 of the heel 50. The plate 55 has a mating surface 56. The plate 55 is preferably about a half as thick as the thickness of the heel 50 at the first end 51 of the heel 50.

The mating surface 56 of the plate 55 of the heel can be adhesively connected to the mating surface 26 of the plate 25 of the forefoot 20. Such a connection is preferably permanent.

A heel spring 60 is at the first end 51 of the heel 50. The heel spring 60 has a top 61, a bottom 62, a front 63 and a rear 64. The heel spring 60 generally has an undeflected shape of a semicircle. Heel spring 60 is a rearward opening heel spring, or rearward facing heel spring. The heel spring 60 could have a different shape without departing from the broad aspects of the present invention. During a downward load condition, the heel spring 60 compresses and deforms from its nondeflected semicircular shape in response to the applied force. Conversely, during an upward load condition, the heel spring 60 expands and deforms in response to the applied force. The heel spring can twist during inversion and eversion.

A heel strike is at the second end 52 of the heel. The heel strike 70 has a front end 71 and a rear end 72. The rear 72 of the heel strike comprises a lip 75. The rear end 72 of the heel strike is the rearmost portion of the prosthetic foot 10. The heel strike 70 has a top surface 74 and a bottom surface 75. The top surface 74 of the heel strike 70 coincides with the first surface 53 of the heel 50, and the bottom surface 75 of the heel strike 70 coincides with the second surface 54 of the heel 50. The front 71 of the heel strike 70 is preferably integral with the bottom rear end of the heel spring 60. The body of the heel strike 70 flexibly depends rearward and downwardly from the heel spring 60. The heel strike is generally flat except for the lip during a zero load condition. The lip 73 is generally bent upwards in relation to the remainder of the heel strike 70. During a downward load condition, the heel strike 70 flexes and deflects upwards in response to the applied force.

In the preferred embodiment, the toe spring is located rearward of the heel spring. Further, looking again as FIGS. 5-7, it is shown that either the front 71 of the heel strike or the back bottom end of the heel spring 60 has a width that is narrower than the width of the slit 45 in the toe plate 40. In this regard, the heel 50 passes or extends through the forefoot 20. It will be understood that in an alternative embodiment (not shown) the heel could have a slit and the toe plate could pass through the slit in the heel without departing from the broad aspects of the present invention.

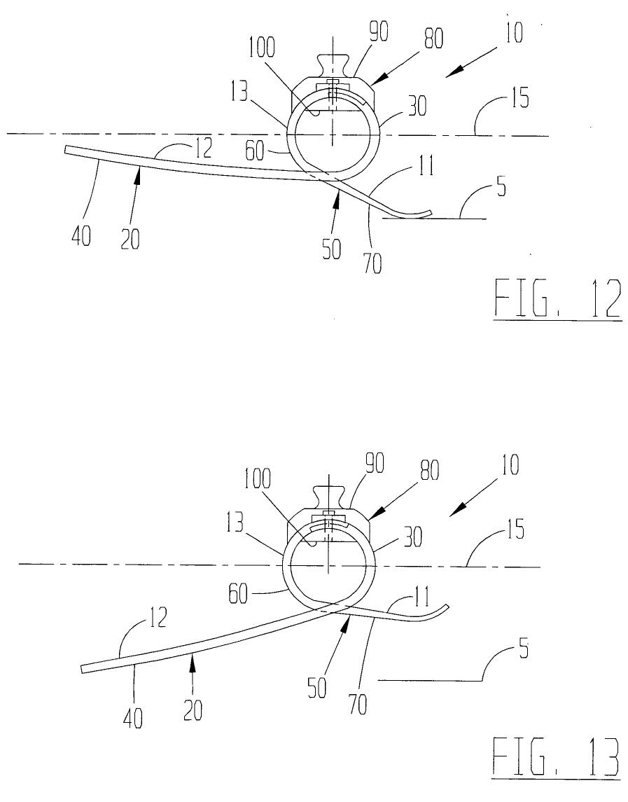

Turning now to FIGS. 8 and 9, a preferred connector 80 is provided. The connector 80 has a top piece 90 and a bottom piece 100. The top piece 90 has a base 91 with a bottom 92 and a top 93. The bottom 92 is preferably concave and complementary shaped to the generally loop shaped foot spring 13. According to one preferred embodiment, the top 93 can have a pyramidal adapter 94 thereon. A first tab 95 with a first hole 96 there through is on one side of the top 90. A second tab 97 with a second hole 98 there through is on the side of the top 90 that is opposite of the side with the first tab 95.

A bottom piece 100 is also provided. The bottom piece 100 has a bottom surface 101 and a top surface 102. The bottom surface 101 is preferably flat. The top surface is preferably convex and complementary to the generally loop shaped toe spring 13. Holes 103 are provided for receiving bolts 105. The holes 103 are spaced apart a distance equal to the distance between holes 96 and 98 of the top piece 90. In this regard, the top and bottom pieces 90 and 100 can be aligned and bolted together using bolts 105. The connector 80 has an alignment axis 106. The bolts 105 are inserted into holes 103 generally parallel to the alignment axis 106.

Looking now to FIG. 10, an alternative preferred embodiment of the connector top 110 is provided. While not shown, it will be understood how to modify the connector bottom to coact with this preferred connector top. In this regard, the connector 110 has a top piece 120 with a base 121 that is complementary to the foot spring 13. The top piece 120 further has a pyramidal adapter 124 on its top. A first tab 125 having two holes 126 there through is on one side of the top 120. A second tab 127 with a two additional holes 128 there through is on the side of the top 120 that is opposite of the side with the first tab 125.

Looking now to FIG. 11, a further alternative preferred embodiment of the connector top 130 is provided. Connector 130 can preferably be bolted to bottom piece 100. The connector 130 has a top piece 140 with a base 141 that is complementary to the foot spring 13. The top piece 140 further has a receiver adapter 144 on its top. A first tab 145 having a hole 146 there through is on one side of the top 140. A second tab 147 with a second hole 148 there through is on the side of the top 140 that is opposite of the side with the first tab 145.

Turning now to FIGS. 2 and 3, it is shown that the connector 80 is complementarily connected to the foot spring 13 of the prosthetic foot 10 of the present invention. In this regard, the bolts 105 are used to clamp the connector 80 onto a selected position on the foot spring 13. The connector is frictionally held in its selected position on the foot spring 13.

The connector can be selectively positioned on the foot spring 13 to selectively adjust the location of the heel strike 70 with respect to the connector 80. One result is that the overall length of the prosthetic limb can be adjusted by adjusting the orientation of the alignment axis 106 of the connector on the foot spring 13. Two examples are provided, and are shown in FIGS. 12 and 13. In FIG. 12, the connector is adjusted to a forward position. In the forward position, the heel strike 70 is lowered in relation to the remainder of the foot 10. Also, the distance between the heel strike 70 and the connector 80 is increased when the connector 80 is rotated forward on the foot spring 13. Conversely, as shown in FIG. 13, the connector 80 can be adjusted rearward to a rearward position. In the rearward position, the heel strike 70 is raised in relation to the remainder of the foot 10. Also, the distance between the heel strike 70 and the connector 80 is decreased when the connector 80 is rotated rearward on the foot spring 13. It will be understood that the connector 80 can be adjusted to any desired location on the foot spring 13.

Operation of the present invention is illustrated in FIGS. 14-16. FIG. 14 is illustrative of loading conditions at heel-strike. At heel-strike, the heel strike 70 makes initial contact with the ground 5, and the front foot portion 12 is off the ground completely. The heel strike 70 flexibly deflects upward and the heel spring 60 compresses. The deflection of the heel strike 70 is severe, especially when the person is heavy or when the person runs or jumps, or otherwise has a lot of momentum that needs to be absorbed by the foot 10. Energy is stored in the deflected heel strike 70 and heel spring 60. The toe plate 40 is not deflected during in heel-strike, but may be moved slightly. The person's weight acting rearward against the connector 80 applies a force that tends to pull the toe plate 40 upwards. The amount of force being transmitted to the foot spring 13 will tend to pull the tow plate 40 downwards. The toe plate 40 will move up or down depending on which force is greater.

As the person moves toward mid-stance, the heel strike 70 releases some of its stored energy to assist in propelling the foot 10 to the mid-stance position. Also, the heel spring 60 releases some of its stored energy to assist in raising the person upwards.

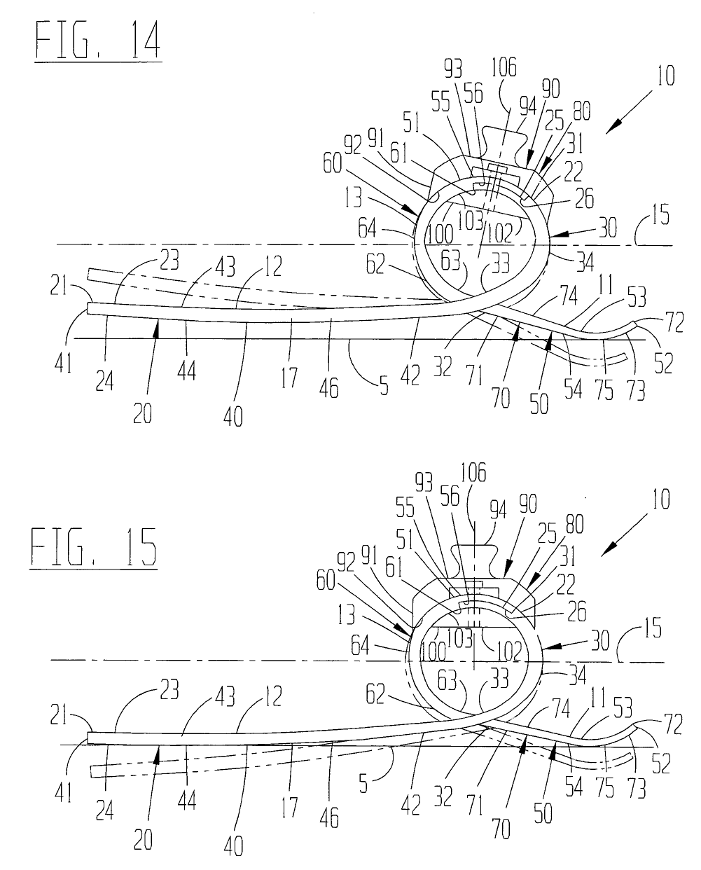

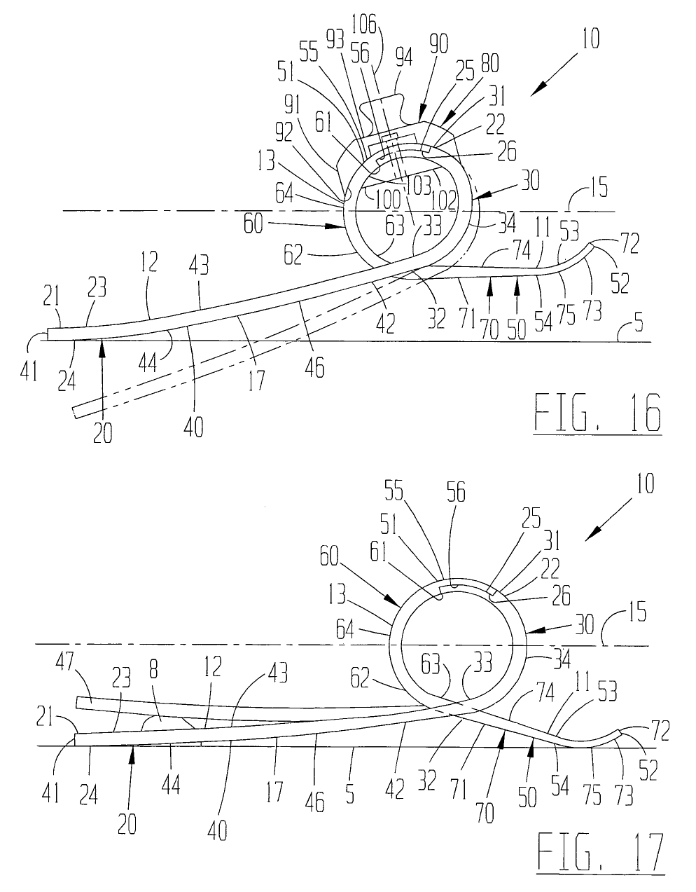

An operational view at mid-stance is provided in FIG. 15. At mid-stance, the heel strike 70 and the toe plate 40 both contact the ground, and are both moderately deflected. Further, the foot spring 13 is moderately compressed. If the person chooses to rock back onto the heel strike 70, the toe spring 30 portion of the foot spring 13 decompresses, or expands, and the toe plate deflexes to assist the person in rocking backwards. Conversely, if the person chooses to move towards toe-off, the heel strike 70 and the heel spring 60 portion of the foot spring 13 release energy to assist the person towards toe-off.

It is noteworthy, that if the person happens to land in a flat foot orientation, the toe plate 40 and the heel strike 70 may deflect severely, and the foot spring 13 may compress severely, while absorbing the shock, and then release some energy to return the foot to mid-stance equilibrium.

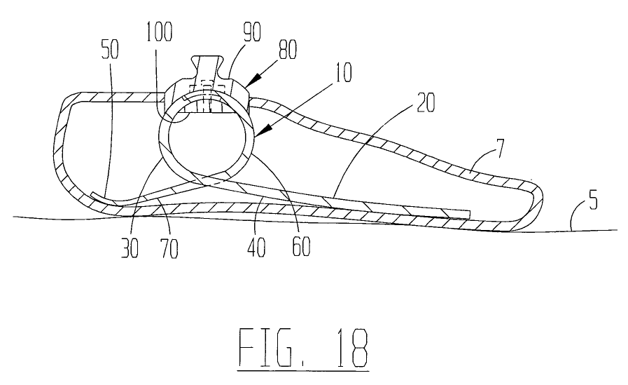

FIG. 16 shows an operational view of the present invention at toe-off. At toe-off, the front end 41 of the toe plate 40 is the only part of the foot that is contacting the ground 5. The toe plate 40 is severely deflected and foot spring 30 is compressed. The entire rear foot portion 11 is slightly moved forward with respect to the toe plate 40 at toe-off, due to the compression of the foot spring 13. The heel strike 70 is not deflected during toe-off. The toe plate 40 releases energy during toe-off that pushes the foot in a direction generally perpendicular to the bottom surface 44 of the toe plate 40.

Thus it is apparent that there has been provided, in accordance with the invention, a prosthetic foot that fully satisfies the objects, aims and advantages as set forth above. While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims.

I claim:

1. A prosthetic foot comprising:
at least one toe;
a heel strike;
a foot spring that is generally loop shaped when undeflected and that interconnects said at least one toe and said heel strike; and
a connector connectable to said foot spring for connecting said foot to a prosthetic component.

2. The prosthetic foot of claim 1 wherein said foot spring comprises a forward opening toe spring integral with said at least one toe and a rearward opening heel spring integral with said heel strike.

3. The prosthetic foot of claim 2 wherein at mid-stance, said at least one toe flexes upwards, said heel strike flexes upwards, said toe spring compresses and said heel spring compresses such that said foot spring constricts in size from its undeflected general loop shape.

4. The prosthetic foot of claim 2 wherein:
said heel spring has a heel spring plate with a heel spring plate mating surface;
said toe spring has a toe spring plate with a toe spring plate mating surface; and
said heel spring plate mating surface is adhesively connected to said toe spring plate mating surface.

5. The prosthetic foot of claim 2 wherein:
said at least one toe comprises a first toe and a second toe separated by a slit having a selected width, and
one of said heel strike and said heel spring passes through said slit.

6. The prosthetic foot of claim 2 wherein:
said forward opening toe spring has a rear;
said rearward opening heel spring has a front; and
said front of said rearward opening heel spring is forward of said rear of said forward opening toe spring.

7. The prosthetic foot of claim 1 wherein said connector has a connector surface that is contactable against said foot spring and is complementary to said generally loop shape of said foot spring, and is an adjustably positionable on said foot spring, whereby adjustment of the position of said connector on said foot spring adjusts the distance from said heel strike to said connector.

8. The prosthetic foot of claim 7 wherein said connector has a top piece and a bottom piece, said top piece and said bottom piece being selectively bolted together to clamp onto said foot spring and frictionally hold said connector in said selected position on said foot spring.

9. The prosthetic foot of claim 7 wherein said top piece of said connector comprises a pyramidal adapter for connecting to a prosthetic component.

10. A prosthetic foot comprising:
a forefoot having a toe plate and a forward opening toe spring;
a heel having a heel strike and a rearward opening heel spring, wherein said rearward opening heel spring and said forward opening toe spring form a foot spring; and
a connector connected to said prosthetic foot for connecting said prosthetic foot to a prosthetic component.

11. The prosthetic foot of claim 10 wherein said foot spring is generally loop shaped when undeflected.

12. The prosthetic foot of claim 11 wherein at mid-stance, said toe plate flexes upwards, said heel strike flexes upwards, and said foot spring constricts in size from its undeflected general loop shape.

13. The prosthetic foot of claim 11 wherein said connector has a connector surface that is contactable against said foot spring and is complementary to said generally loop shape of said foot spring, and is an adjustably positionable on said foot spring, whereby adjustment of the position of said connector on said foot spring adjusts the distance from said heel strike to said connector.

14. The prosthetic foot of claim 13 wherein said connector has a top piece and a bottom piece, said top piece and said bottom piece being selectively bolted together to clamp onto said foot spring and frictionally hold said connector in said selected position on said foot spring.

15. The prosthetic foot of claim 14 wherein said top piece of said connector comprises a pyramidal adapter for connecting to a prosthetic component.

16. The prosthetic foot of claim 10 wherein:
said forefoot has a rear end and has a forefoot mating surface at said rear end of said forefoot;
said heel has a front end and has a heel mating surface at said front end of said heel; and
said heel mating surface is adhesively connected to said forefoot mating surface.

17. The prosthetic foot of claim 10 wherein:
said toe plate comprises a first toe and a second toe separated by a slit in said foot plate, said slit having a selected width;
said first toe and said second toe are independently deflectable; and
one of said heel strike and said rearward opening heel spring passes through said slit in said toe plate.

18. The prosthetic foot of claim 10 wherein:
said forward opening toe spring has a rear;
said rearward opening heel spring has a front; and
said front of said rearward opening heel spring is forward of said rear of said forward opening toe spring.

19. A prosthetic foot comprising:
a forefoot having a toe plate and a toe spring;
a heel having a heel strike and a heel spring, wherein said heel spring and said toe spring comprise a foot spring; and
a variably positionable connector having a top and being connected to said foot spring for connecting said prosthetic foot to a prosthetic component,
whereby adjustment of said variably positionable connector on said foot spring determines the distance from said heel strike to said top of said variably positionable connector.

20. The prosthetic foot of claim 19 wherein:
said heel spring is a rearward opening heel spring and said toe spring is a forward opening toe spring; and
said foot spring is generally loop shaped when undeflected.

21. The prosthetic foot of claim 20 wherein at mid-stance, said toe plate flexes upwards, said heel strike flexes upwards, and said foot spring constricts in size from its undeflected general loop shape.

22. The prosthetic foot of claim 20 wherein said variably positionable connector has a connector surface that is contactable against said foot spring and is complementary to said generally loop shape of said foot spring.

23. The prosthetic foot of claim 22 wherein said variably positionable connector has a top piece and a bottom piece, said top piece and said bottom piece being selectively bolted together to clamp onto said foot spring and frictionally hold said variably positionable connector in said selected position on said foot spring.

24. The prosthetic foot of claim 23 wherein said top piece of said variably positionable connector comprises a pyramidal adapter for connecting to a prosthetic component.

25. The prosthetic foot of claim 20 wherein:
said forward opening toe spring has a rear;
said rearward opening heel spring has a front; and
said front of said rearward opening heel spring is forward of said rear of said forward opening toe spring.

26. The prosthetic foot of claim 19 wherein:
said forefoot has a rear end and has a forefoot mating surface at said rear end of said forefoot;
said heel has a front end and has a heel mating surface at said front end of said heel; and
said heel mating surface is adhesively connected to said forefoot mating surface.

27. The prosthetic foot of claim 19 wherein:
said toe plate comprises a first toe and a second toe separated by a slit in said foot plate, said slit having a selected width;
said first toe and said second toe are independently deflectable; and
one of said heel strike and said heel spring passes through said slit in said toe plate.
